(19) United States
(12) Patent Application Publication
(10) Pub. No.: US 2015/0265388 A1
(43) Pub. Date: Sep. 24, 2015

(54) SYSTEM AND METHOD FOR TREATMENT OF ANAL AND FECAL INCONTINENCE

(71) Applicant: AMS RESEARCH CORPORATION, Minnetonka, MN (US)

(72) Inventors: Carrie L. Herman, Mayer, MN (US); Amanda J. Heys, Eden Prairie, MN (US); Chaouki A. Khamis, Cupertino, CA (US); Peter L. Rosenblatt, Newton, MA (US)

(21) Appl. No.: 14/437,103

(22) PCT Filed: Oct. 22, 2013
(86) PCT No.: PCT/US13/66143

§ 371 Date: Apr. 20, 2015

Related U.S. Application Data
(60) Provisional application No. 61/716,815, filed on Oct. 22, 2012.

(51) Int. Cl. A61F 2/00; A61B 17/34

(52) U.S. Cl. CPC A61F 2/0045; A61B 17/3468; A61F 2220/0016

(57) ABSTRACT

Described are implants, systems, kits, and methods useful for treating fecal incontinence, which may involve surgical implantation of the implant to place a tissue support portion at a location to support tissue of a region of anal musculature, with extension portions placed in a position to support the tissue support portion. A needle delivery device can be included to implant the implant.

### PRIORITY CLAIM

The present non-provisional patent application claims priority under 35 USC §119(e) from U.S. Provisional Patent Application having Ser. No. 61/716,815, filed on Oct. 22, 2012, by Herman et al., titled “SYSTEM AND METHOD FOR TREATMENT OF ANAL INCONTINENCE AND PELVIC ORGAN PROLAPSE”, wherein the entirety of said provisional patent applications are incorporated herein by reference.

### FIELD OF THE INVENTION

The invention relates to apparatus and methods for treating fecal incontinence by use of a pelvic implant to support the rectum or anal sphincter. In particular, the present invention relates to a surgically implanted fecal implant that support the rectum and that is secured to pelvic tissue to provide that support.

### BACKGROUND

Men, women, and children of all ages can suffer from fecal incontinence and loss of bowel control. Their lives are perpetually interrupted by thoughts of ensuring that they have ready access to a restroom. Everyday activities such as attending a theater or sporting events can become unpleasant. Sufferers often begin to avoid social situations in an effort to reduce the stress associated with their condition.

A variety of treatment options are currently available for treating fecal incontinence. Some of these include external devices, behavioral therapy (such as biofeedback, electrical stimulation, or Kegel exercises), prosthetic fecal sphincters (including the ACTICON® Neosphincter available from American Medical Systems), and surgery. Depending on the age, medical condition, and personal preference of a patient, surgical procedures can be used to completely restore continence.

Example of implants are described in U.S. Pat. Nos. 5,112,344; 5,899,909; 6,039,686; 6,042,534; 6,110,101; 6,478,727; 6,638,211; and PCT Publication Nos. WO 02/39890 and WO 02/069781, which are herein incorporated by reference.

Other methods for treating pelvic conditions involve installation of an implant below the anus through incisions made at posterior to the anus. See, e.g., U.S. Pat. No. 6,911,003 and U.S. Publication No. 2003/0171644A1, the entireties of which are incorporated herein by reference.

While present methods of treating incontinence (e.g., fecal incontinence) can be effective, safe, and long-lasting, there is ongoing effort toward improving these methods.

### SUMMARY

The present patent application describes pelvic implants and methods for treating fecal and anal incontinence. Embodiments of implants include an implant with a self-fixating tip at a distal end of one or more extension portions. The self-fixating tip can be placed at and secured within internal tissue of the pelvic region to support the extension portions and pelvic tissue that is supported by the implant. As an example, a self-fixating tip can be placed at tissue of the levator ani (this phrase referring to the puborectalis muscle, pubococcygeous muscle, and illiococcygeous muscle). The self-fixating tips can be designed to engage a distal end of an insertion tool to allow the insertion tool to place the self-fixating tip at a desired tissue location by pushing.

As a single example of a useful placement of a fecal implant, a tissue support portion can be placed at a position posterior to the anal sphincter, which includes, e.g. tissue near the anal canal, central tendon, or the levator ani muscle (including puborectalis, pubococcygeous, and illiococcygeous) or at or near a combination of two or more of these tissues.

The implant can include a tissue support portion that supports tissue of a region of anal musculature such as the anal sphincter, and also can include an extension portion that connects to the tissue support portion and that attaches to a different location of the patient's anatomy to support the tissue support portion and, in turn, tissue of a region of anal musculature.

The extension portion can be placed at any location to provide support for the tissue support portion to improve anal continence. As an example, an extension portion can lead to the levator ani muscles and be attached internally to the puborectalis, pubococcygeous, or illiococcygeous.

According to specific embodiments that place an end portion at or through a tissue such as puborectalis, pubococcygeous, or illiococcygeous, an implant can include two opposing elongate end portions that extend to and optionally pass to or through the tissues of the levator ani. The tissue support portion can function to support or approximate tissue of a region of anal musculature, such as the anal sphincter. For example, tension can be placed on the implant to support or approximate one or more of the external anal sphincter, internal anal sphincter, central tendon, anal canal, and rectum. One possible result can be to recreate, support, or reposition, the ano-rectal angle in a manner that improves fecal continence. In a related or alternate embodiment, the implant or mesh can be used to reposition the rectum and the surrounding tissues and organs to establish normal alignment of the anal and rectal canal and colon, thereby bringing about the normal location of the various pelvic components found in younger or continent males and females. According to exemplary embodiments, a central support portion of the implant can be placed in contact with or adhered to tissue of a region of anal musculature, e.g., the anal sphincter, as described, and optionally being secured to or adhered to one or more of these tissues. The implant can be tensioned to support pelvic tissue to improve fecal continence.

According to certain exemplary methods, a surgical installation can involve a posterior incision, wherein the posterior incision is medical anus, or wherein the posterior incision is two incisions lateral to the anus. Tension can be applied to the extension portions of the implant to approximate rectal or anal tissue (e.g., anal sphincter) to restore the normal ano-rectal angle.

Such methods may be useful to treat patients with spinal cord injuries, after spinal cord damage and surgery, in paraplegics, and in patients who suffer from congenital spinal cord disorders such as spina bifida leading to stool incontinence.

An implant can be installed using one or more tools to manipulate the implant to a desired position. Examples include curved two-dimensional tools shaped to allow placement of an implant through a posterior incision. Examples of these types of tools are shown, e.g., at U.S. Patent Publication Numbers 2010/0105979 and 2013/0006048, which are incorporated herein by reference.

According to exemplary methods, an implant can be inserted through a posterior incision that allows access to levator tissue, optionally with some amount of dissection. The incisions can be a variety of incisions that provide such access, such as a small posterior incision that can allow a tissue path to extend from the external posterior incisions to levator tissue or a Kraske incision under the rectum. An implant or a portion of the implant can be accessed or placed into position using a posterior incision.

An exemplary method includes making a posterior incision posterior to the anus. A posterior incision can mean one incision located medial and posterior to the anus, or alternatively, two incisions located lateral to a midline posterior to the anus. The incisions can be, optimally, orientated vertically (meaning a superior to inferior orientation) or, alternatively horizontally (meaning a left to right orientation). A device for aiding in keeping the incisions open is also contemplated. Such as aid can adhere to the patient's skin to keep the incisions open.

Also contemplated according to the present description are kits useful for commercial sale to surgeons, that include an implant and an insertion tool adapted to install the implant using a method as described, such as a posterior incision method. The implant and the tool can be specifically designed to be useful for a single posterior incision method or a method utilizing two posterior incisions lateral to a mid-line, in that the tools can be designed with specific features of strength, and the implant can exhibit properties of increased strength, increased area of contact between the central support portion and tissue, and increased short-term and long-term fixation upon installation.

In one aspect, the invention relates to a method of treating fecal or anal incontinence. The method includes: providing a support portion and an extension portion, creating an incision posterior to the anal sphincter, creating a tissue path inferior to the levator ani, from the incision to a region of levator ani, placing the tissue support portion to support tissue of the region of anal sphincter, and extending the extension portion to a location to support the tissue support portion in a manner that improves fecal continence.

In another aspect the invention relates to a pelvic implant assembly and systems and kits that contain the implant assembly. An example implant assembly includes: an implant comprising a support portion and two extension portions, and a self-fixating tip connected at an end of each extension portion. An alternative example implant assembly includes: two implants each comprising a support portion, an extension portions, and a self-fixating tip connected at each end of the extension portion. Each self-fixating tip includes: a base comprising a proximal base end and a distal base end, the proximal base end being connected to the extension portion. The base includes an internal channel extending from the proximal base end at least partially along a length of the base toward the distal base end, and a fixed lateral extension extending from the base. The total length of one implant is sufficient for the implant to reach from a region of the levator ani, through a tissue path leading to tissue of a region of anal musculature, and through a tissue path leading to an opposite region of the levator ani tissue. The total length of another implant is sufficient for the implant to reach from a region of the levator ani, through a tissue path leading to a tissue of a region of anal musculature.

Potential advantages related to the use of the certain of the currently-described methods and devices can include reduced overall trauma of a procedure due to one or more of: design of a self-fixating tip or insertion tool; reduced trauma caused by a self-fixating tip or associated insertion tool, due to a reduced length of tissue passages; reduced trauma based on the ability to avoid tissue passages next to critical structure; and reduced trauma due to the ability to eliminate the need for local stab (external) incisions otherwise required for needle entry and exit sites.

According to exemplary methods, a physician identifies tissue within the pelvic region to which a self-fixating tip will be secured. An insertion tool and self-fixating tip can be introduced through a posterior incision to insert a permanent (plastic i.e., polypropylene) or bioresorpable implant assembly that includes a self-fixating tip having one or multiple lateral extension portions, to the target site. This procedure can be performed by use of a single posterior incision or through the use of two posterior incisions.

One embodiment of implant is a fecal incontinence implant that includes a sling body, a first self-fixating tip attached to a first end of the implant, and a second anchor member attached to a second end of the implant, wherein the implant is made of a single piece of mesh material.

In another aspect, the invention relates to a method of treating a pelvic condition such as fecal or anal incontinence. The method includes providing an implant according to the current description; providing an insertion tool that includes a handle and a needle extending from the handle, the needle including a proximal end attached to the handle and a distal end, the distal end including a needle distal end that removably engages the self-fixating tip; engaging the needle distal end with the self-fixating tip, inserting the needle distal end and tip through a posterior incision in a patient; and inserting the self-fixating tip into tissue in the levator ani.

Yet another aspect of the invention relates to a method of treating a pelvic condition. The method includes: creating only one incision posterior to the anal sphincter; providing an implant according to the present description; inserting a self-fixating tip through the incision and anchoring the self-fixating tip within muscular tissue; inserting a second self-fixating tip through the incision and anchoring the self-fixating tip within muscular tissue; positioning the implant into a desired supporting position relative to tissue of the pelvic region; and closing the incision.

Alternatively, the method includes: creating two incisions posterior to the anal sphincter and lateral to the sphincter; providing an implant according to the present description; inserting a self-fixating tip through the incision and anchoring the self-fixating tip within fibrous tissue; inserting a second self-fixating tip through the incision and anchoring the self-fixating tip within fibrous tissue; positioning the implant into a desired supporting position relative to tissue of the pelvic region; and closing the incision.

An aspect of the invention includes a method of treating fecal or anal incontinence wherein a posterior incision is made posterior to the anus. An implant comprising a tissue support portion, extension portion, and self-fixating tip is inserted through the posterior incision with an insertion tool and the self-fixating tip is anchored within the levator ani which suspends the tissue support portion to support the rectum or anal sphincter.

Another aspect of the invention includes providing a kit for the treatment of anal or fecal incontinence. The kit comprises an implant having a tissue support portion, an extension portion and a self-fixating tip and the insertion tool comprises a handle and needle. The implant is adapted to extend from a region of anal musculature to a region of a levator ani to support the anal musculature.

### BRIEF DESCRIPTION OF DRAWINGS

Other features and advantages of the present invention will be seen as the following description of particular embodiments progresses in conjunction with the drawings. Drawings are schematic and not to scale.

FIG. 1 depicts the anatomy of the bony pelvis;

FIG. 2 illustrates a single incision method;

FIG. 3 illustrates a double incision method;

FIGS. 4a-5 shows an exemplary final position of length of supporting material;

FIGS. 6a and 6b show a side view of a final position of length of the implant;

FIGS. 7-8 illustrates an exemplary embodiment of the implant;

FIG. 9 illustrates an exemplary multi-piece implant;

FIG. 10 illustrates an exemplary embodiment of the implant;

FIGS. 11a and 11b illustrate a device for aiding a posterior incision; and

FIG. 12 shows an exemplary insertion tool.

### DETAILED DESCRIPTION

The following description is meant to be illustrative only and not limiting. Other embodiments of this invention will be apparent to those of ordinary skill in the art in view of this description.

The present invention is directed to surgical instruments, assemblies, and implantable articles for treating anal and fecal incontinence. Described are various features of surgical implants, surgical tools, surgical systems, surgical kits, and surgical methods useful for installing implants.

An implant can include a tissue support portion (or “support portion”) that can be used to support pelvic tissue such as the rectum or anal sphincter. During use, the tissue support portion can be placed in contact with and attached to tissue to be supported, for example, the anal sphincter. An implant can additionally include one or more extension portions attached to the tissue support portion. Examples of pelvic implants are described in the following exemplary documents: U.S. Pat. Nos. 7,905,825; 7,722,528; and 7,740,576; the entireties of each of these disclosures being incorporated herein by reference.

An implant may include portions or sections that are synthetic or of biological material (e.g., porcine, cadaveric, etc.). Extension portions may be, e.g., a synthetic mesh such as a polypropylene mesh. The tissue support portion may be synthetic (e.g., a polypropylene mesh) or biologic. Examples of implant products that may be similar to those useful according to the present description, include those sold commercially by American Medical Systems, Inc., of Minnetonka Minn., under the trade names Apogee® and Perigee® for use in treating pelvic prolapse (including vaginal vault prolapse, cystocele, enterocele, etc.), and Sparc®, Bioarc®, and Monarc® for treating urinary incontinence.

Exemplary implants can include one or more tissue support portions for placing in contact with tissue to be supported and one or more extension portions, the tissue support portion being useful to support a specific type of pelvic tissue such as the anus or rectum. The tissue support portion can be sized and shaped to contact the desired tissue when installed, e.g., as a “sling” or “hammock,” to contact and support pelvic tissue. A tissue support portion that is located between two or more extension portions is sometimes referred to herein as a “central support portion” or a “support portion.”

Extension portions are elongate pieces of material that extend from the tissue support portion and either are or can be connected to the tissue support portion, and are useful to attach to anatomical features in the pelvic region (e.g., using a self-fixating tip) to thereby provide support for the tissue support portion and the supported tissue. One or multiple (e.g., one or two) extension portions can extend from the tissue support portion as elongate “ends,” “arms,” or “extensions,” useful to attach to tissue in the pelvic region, such as by extending through a tissue path to an internal anchoring point as described herein.

An example of a particular type of pelvic implant is the type that includes supportive portions including or consisting of a central support portion and either two elongate extension portions extending from the central support portion. An implant that has exactly two extension portions can be of the type useful for treating anal or fecal incontinence. The term “supportive portions” refers to portions of an implant that function to support tissue after the implant has been implanted, and specifically includes extension portions and tissue support portions, and does not include optional or appurtenant features of an implant such as a sheath or self-fixating tip or other type of connector for attaching the implant to an insertion tool.

Types of exemplary implants that can be generally useful as discussed herein can include those previously and currently used in treating pelvic conditions, including those implants referred to as urethral “slings,” “strips,” “mesh strips,” “hammocks,” among other terms for pelvic implants. An exemplary implant can be an integral mesh strip with supportive portions consisting of or consisting essentially of a central support portion and two extension portions. Other exemplary fecal sling implants are described in U.S. Pat. Nos. 7,794,385; 7,828,715; 8,172,745; 8,449,447, which are hereby incorporated by reference.

Dimensions of an implant can be as desired and useful for any particular installation procedure, treatment, patient anatomy, and to support a specific tissue or type of tissue. Exemplary dimensions can be sufficient to allow the tissue support portion to contact tissue to be supported, and to allow extension portions to extend from the tissue support portion to a desired anatomical location to allow the extension portion be secured to anatomy of the pelvic region, to support the tissue support portion.

Dimensions of extension portions according to the invention can allow the extension portion to reach between a tissue support portion placed to support pelvic tissue (at an end of the extension portion connected to the tissue support portion) and a location at which the distal end of the extension portion attaches to pelvic tissue. A distal end of an extension portion, according to embodiments of the invention, can include a self-fixating tip that can be attached directly to pelvic tissue such as levator muscles. The length of the extension portion, therefore, can be in a range that allows placement of a tissue support portion as desired to support pelvic tissue, while the self-fixating tip is installed in pelvic tissue.

As described elsewhere herein, a length of an extension portion can optionally be fixed (i.e., the extension portion does not include any form of length-adjusting mechanism), as can a length of an implant spanning from opposite self-fixating tips and including extension portions and a length or segment of tissue support portion. Alternate embodiments of implants of the invention may include adjustment or tensioning mechanisms that allow a physician to alter the length of an extension portion before, during, or after implantation. On the other hand, adjustment and tensioning mechanisms can also be excluded from embodiments of implants of the invention by selecting the length of extension portions and tissue support portions, and by adjusting for tensioning or positioning of extension portions and tissue support portions based on placement of the self-fixating tip within the pelvic tissue, selected placement including selection of the point of insertion of a self-fixating tip and depth of insertion of the self-fixating tip.

The ability to select a point of insertion and depth of penetration of a self-fixating tip into a tissue is a feature of exemplary self-fixating tips and their methods of use, according to the invention, that allows a surgeon to select a location of an implant, to select an amount of tension placed on an installed implant, to place a desired amount of supportive force on a supported pelvic tissue, or combinations of these. With this feature, embodiments of the invention may avoid the need for a separate length-adjustment or tensioning mechanism, and embodiments of implants according to the invention can optionally exclude any sort of length-adjustment feature or tension-adjustment feature; these features include the use of separate implant pieces that can be secured together as desired to select a length of an extension portion or length of an implant.

According to this exemplary advantageous technique, a physician (e.g., surgeon) is able place an implant between locations at opposite tissues of the levator ani to position the implant to support the anal sphincter, without the need for an adjustment feature designed into the implant. The surgeon inserts a first self-fixating tip in tissue of one levator ani, preferably in the puborectalis muscle, at a desired position and a desired depth. The second self-fixating tip located on the opposite extension portion of the implant can be inserted into tissue of the opposite levator ani, preferably the puborectalis muscle, and the position or tension of the implant below the anus, can be selected, controlled, or adjusted by the depth and placement of the self-fixating tips within the tissue of the opposite levator ani.

Because some exemplary embodiments of implants and their extension portions do not require either a length-adjusting or a tension-adjusting mechanism, these embodiments of implants of the invention can include a fixed length of implant material separating two opposing self-fixating tips. A “fixed” length of material can mean that the implant does not include a length-adjusting feature such as discussed elsewhere herein, but still may exhibit an amount of elasticity or other normal mechanical properties of an implant material. A fixed length of implant material can be of a single piece of material (integral), or may be of multiple pieces secured together in a manner that does not allow further adjustment of the length. For example, multiple pieces of identical mesh material may be assembled into a single implant, before implanting the assembled implant, by sewing or otherwise attaching pieces together. Pieces of different types of mesh materials may be sewn or otherwise secured together, or pieces of synthetic material may be sewn or otherwise secured to a biologic material, in a manner that does not allow for adjustment of dimensions of the assembled implant.

In an alternative embodiment, the implant may include a length-adjusting or tension-adjusting mechanism. A fixed-length support portion can include an adjustability feature wherein extension arms have a length adjusting ability through the adjustability features. In still yet another embodiment, fixed-length extension arms can include an adjustability feature wherein support portion has a length adjusting ability through the adjustability feature.

A self-fixating tip can be connected to an extension portion of an implant in any fashion, directly by any attachment mechanism, or indirectly such as through an attachment structure such as a suture. A connection can be based on a mechanical structure, by adhesive, by a connecting suture, or by an integral connection such as by injection molding or “insert” molding (also, “over molding”) as described in U.S. Publication No. 2006/0260618, incorporated herein by reference. According to that description a thermoplastic or thermosetting polymer material can be insert molded or injection molded at an end of a mesh extension portion of an implant, e.g., directly to the mesh. By this method, a molded polymer can form a self-fixating tip at an end of an extension portion. The self-fixating tip can be as described herein, for example, including lateral extensions and an internal channel.

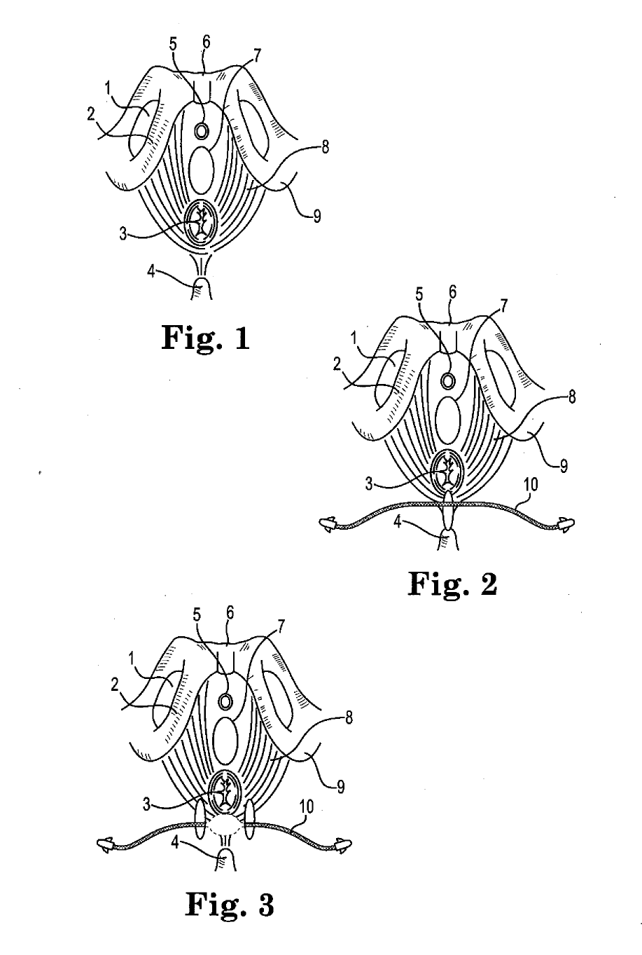

FIG. 1 shows the anatomy of the bony pelvis, with the pubic symphysis (6), the ischiopubic ramus (2), the ischial tuberosity (9), the coccyx (4), and the obturator foramen (1). It also demonstrates the relationship of the levator ani muscles (8) (and, in particular, the puborectalis) to the urethra (5), vagina (7), and rectum (3).

Although embodiments of the present invention have been described with reference to the treatment of anal or fecal continence, it should be appreciated that many of these embodiments would also be suitable to repair a variety of pelvic conditions in both males and females. For example, embodiments of the present invention would be suitable for a variety of pelvic floor repairs and/or treatments, including pelvic organ prolapse repair, levator hiatus repair, urinary incontinence treatment, perineal body support and hysterectomy support.

An example of a method according to the invention is a method of treating anal incontinence by surgical implantation of an implant (e.g., a single, integral, optionally uniform, woven polymeric mesh strip, with two self-fixating tips, one on each end) through a posterior incision, along a tissue path that extends from a region of the anus to the levator ani. These methods can advantageously involve only a single incision as shown in FIG. 2 and can exclude the need for any additional incisions. An elongate implant is attached at tissue of the opposing levator ani by self-fixating tips at opposing distal ends of the implant, with the implant positioned to pass below the rectum to support the anal sphincter.

FIG. 3 illustrates an alternative method according to the invention. A method using two posterior incisions includes creating two posterior incisions posterior and lateral to the anal sphincter. The implant 10 and self-fixating tip 16a is inserted through the first incision and anchored within muscular tissue while the support portion 14 is pulled through the second incision and the second self-fixating tip 16b is anchored with muscular tissue.

The implant may be placed using one or more insertion tools as described, by installing extension portions of the implant between the incision and the levator ani, with the support portion of the implant positioned below the rectum. The extension portions may be pushed through the tissue path at the lead of an insertion tool that engages the self-fixating tip. The tissue support portion of the implant may be placed as desired to support the rectum or anal sphincter, optionally with approximation, compression, or a combination of approximation and compression. Adjustment of the implant can be performed based on the location (point of entry) and depth of insertion of the self-fixating tips at tissue of the opposing levator ani muscles. The implant may optionally include a widened central support portion that is placed to contact the rectum or anal sphincter, and the support portion and implant are used to approximate the anal sphincter to improve continence.

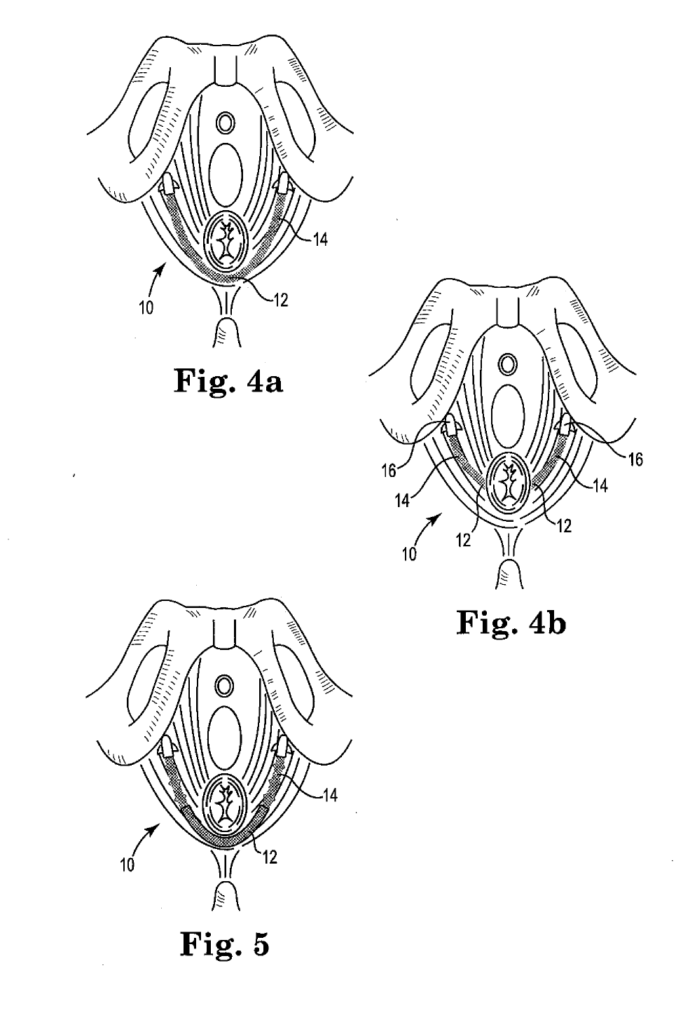

FIG. 4a demonstrates a final position of the synthetic mesh 10 under the anus and/or rectum, with the incision between the anus and the coccyx, and up through the medial portion of the levator ani. Using female anatomy as an example, an insertion tool and self-fixating tip can be inserted in to the levator ani along a tissue path from the posterior incision to the levator ani. Based on that tissue path, and the direction of fibers of the levator ani muscle, it has been found that an insertion tool that includes a curve (in two dimensions) that allows the distal end to be located at the levator ani muscle when the needle is inserted through a posterior incision.

In another method, as shown in FIG. 4b, an alternative implant 10 is provided. Implant 10 comprises two pieces, each piece having a tissue support portion 12, an extension portions 14, and a self-fixating tip 16. Tissue support portion 12 is located at an opposite end of the extension portion 14 as the self-fixating tip 16. The tissue support portions 12 are positioned at the anal sphincter and the extension portions 14 extend to the levator ani where the self-fixating tips 16 are anchored. The support portion 12 may be attached to the anal sphincter or adjacent tissue e.g., by suture, anchor, or other structure or method known to those in the art.

In one embodiment of a method of the invention, an implant such as implant 10 may be introduced using an “inside-out” approach to place the implant below the rectum with ends at tissue of the two opposite levator ani tissue, through a posterior incision. The precise, final location of an implant will depend on a variety of factors including the particular surgical procedure performed, and any preconditions of the patient such as scar tissue or previous surgeries. For example, it may be preferred to place an implant such as implant 10 in close proximity to, but not in contact with, a mid portion of the anus to treat incontinence.

For a typical procedure for treating any pelvic condition, a patient may be first placed under local, spinal, or general anesthesia. For implantation of an implant 10 to treat fecal incontinence, a posterior incision is made posterior to the anus. The incision should be large enough for the surgeon to place the implant 10 through the incision using selected instruments. A desired amount of tissue may optionally be dissected inferior to the levator ani on each side, for placement of implant 10. In one embodiment the tissue may be dissected approximately 1-2 centimeters in each direction away from the anus and toward opposing locations for self-fixating tips 16 (e.g., at tissue of the levator ani). The dissection of tissue may be as much or as little as desired, including none. The first self-fixating tip 16 is then placed through the incision and directed toward the desired anchoring position (e.g., tissue of the levator ani).

FIG. 5 demonstrates a implant made of a synthetic material such as silastic or other plastic with serrations on each arm that maintain the implant in position after adjustment by the surgeon.

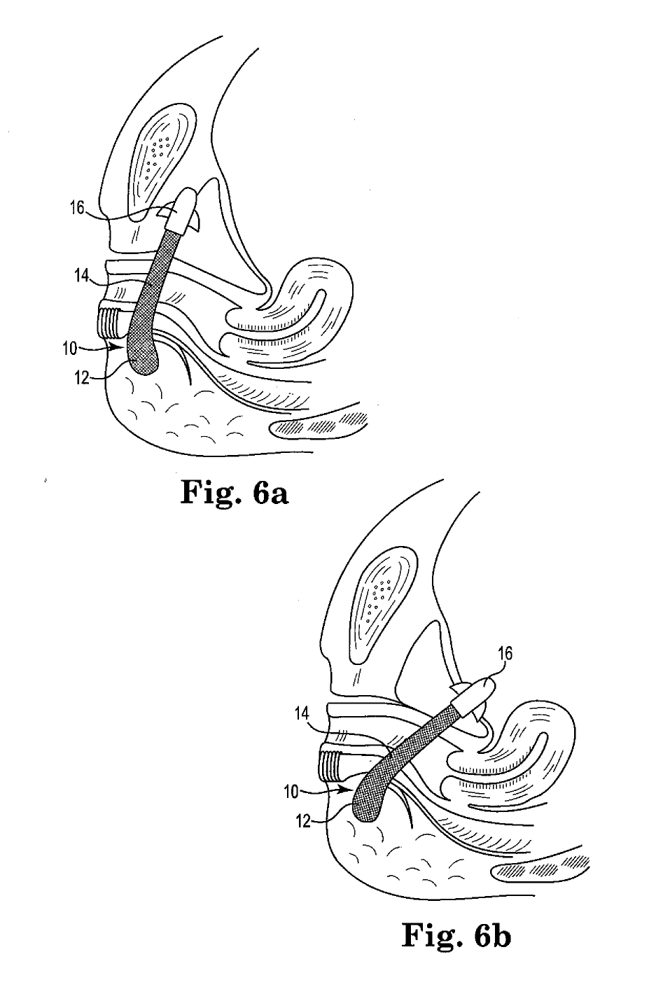

FIG. 6 shows an implant 10 oriented in a lateral orientation of the pelvis. The self-fixating tip 16 is anchored within tissue of the levator ani, while the support portion 12 is supporting the rectum.

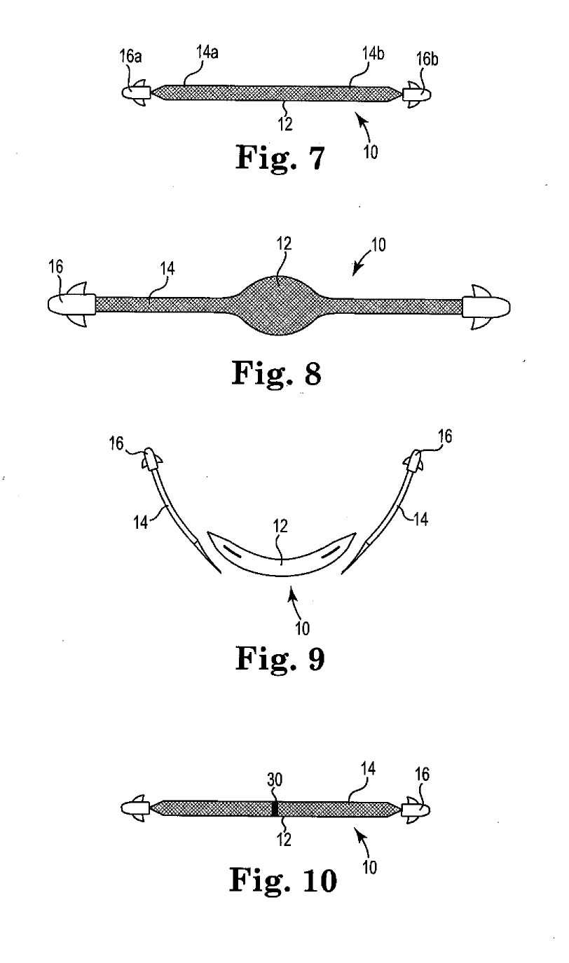

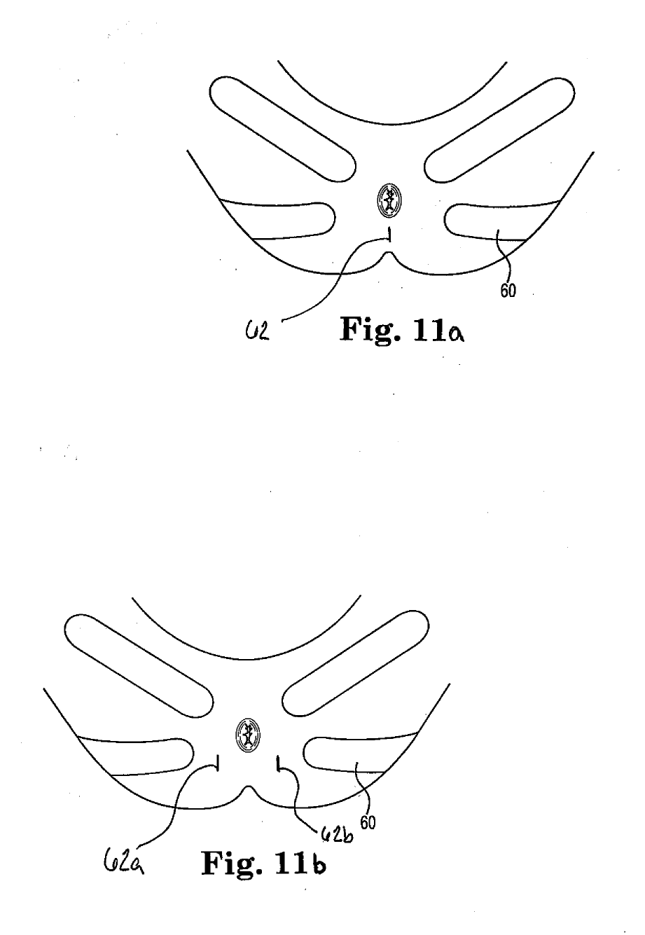

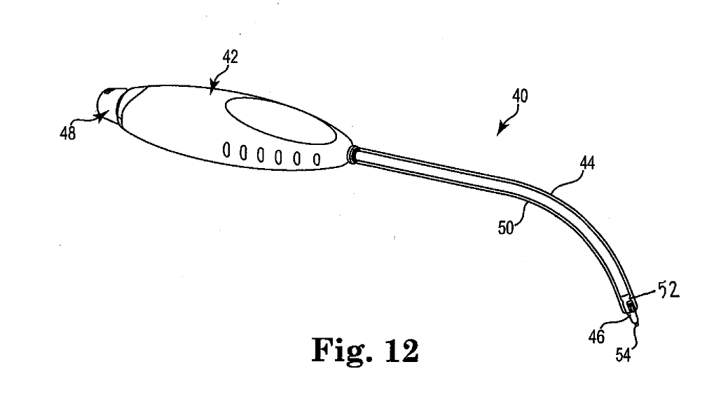

FIGS. 7-10 illustrate an implant 10 comprising a tissue support portion 12 and at least one extension portion 14. The one or more extension portion 14 may have a self-fixating tip 16 operatively coupled to the distal end of the extension portion 14. The self-fixating tips 16a, 16b may be placed through or into tissues of the levator ani. FIG. 10 illustrates the implant of FIG. 7 having indicia 30 at or near the middle portion of the implant 10.

The length of a implant 10, including tissue support portion 12, between distal ends of extension portions 14a, 14b, can be sufficient to place opposing self-fixating tips at positions and depths of tissue of the levator ani, with the implant reaching between the opposing levator ani while supporting the rectum. Exemplary lengths of an implant or implant portion for extension below the anus, between opposing levator ani, from distal end to distal end of the extensions while laying flat, can be in the range from about 6 to 15 centimeters, e.g., from 7 to 10 centimeters or from 8 to 9 centimeters or about 8.5 centimeters. (Lengths L1 and L2 of FIG. 7 can be within these ranges.) A width of the extension portion can be as desired, such as within the range from about 1 to 1.5 centimeters.

An extension portion 14 of a implant 10 as shown in FIG. 7 of the invention can include a self-fixating tip 16 at an end of the extension portion 14 that is distal from a tissue support portion 12. The self-fixating tip 16 in general can be a structure connected to a distal end of an extension portion 14 and that can be implanted into tissue in a manner that will maintain the position of the self-fixating tip 16 and the attached implant 10. Exemplary self-fixating tips 16 can also be designed to engage an end of an insertion tool 40 so the insertion tool 40 can be used to push the self-fixating tip 16 through tissue for implantation. The self-fixating tip 16 may engage the insertion tool 40 at an internal channel of the self-fixating tip, at an external location such as at the base, or at a lateral extension, as desired.

A self-fixating tip 16 can be made out of any useful material, generally including materials that can be molded or formed to a desired structure and connected to or attached to an end of an extension portion of an implant. Useful materials can include plastics such as polyethylene, polypropylene, and other thermoplastic or thermoformable materials, as well as metals, ceramics, and other types of biocompatible and optionally bioabsorbable or bioresorbable materials. Exemplary bioabsorbable materials include, e.g., polyglycolic acid (PGA), polylactide (PLA), copolymers of PGA and PLA.

A self-fixating tip also, preferably, includes one or more lateral extensions that can increase the force required to remove the self-fixating tip from tissue after insertion into the tissue, i.e. the “pullout force.” At the same time, the lateral extensions can be designed to exhibit a reduced or relatively low “insertion force,” which is the amount of force used to insert the self-fixating tip into tissue. The self-fixating tip is designed to be essentially permanently placed upon insertion into tissue, with the single exception that if absolutely necessary to provide desired placement of the self-fixating tip or an attached implant, the self-fixating tip may be removed by a surgeon during an implantation procedure. The self-fixating tip, and all components of the self-fixating tip, can be of combined form and dimensions to result in these functional features.

Exemplary self-fixating tips discussed herein include a cylindrical base or tapered cylindrical base, with a hollow or solid interior. Other shapes for a base may also be useful, such as blocks having square or rectangular forms when viewed in cross section along a longitudinal axis extending from a proximal base end to a distal base end. For those types of self-fixating tips, dimensions of a square or rectangular cross section can be of a range similar to the diameter of a cylindrical base, such as from about 2 to about 5 millimeters in either dimension when viewed in cross section.

As an example of a specific range of a length of a self-fixating tip, lengths (measured from the proximal base end to the distal base end along a longitudinal axis of the self-fixating tip) in the range from 0.4 to 1.0 centimeter, e.g., from 0.4 to 0.8 centimeters, or from 0.4 to 0.7 centimeters, have been found to be useful. These ranges are specifically useful for self-fixating tips that can be inserted into muscle of the levator ani, because the relatively short length can allow the self-fixating tip to be inserted into the muscle tissue a desired depth, i.e., over a range of depths.

Exemplary lateral extensions can be rigid or “fixed” relative to the base so the lateral extension does not substantially move or deflect during or after implantation. For example, a fixed lateral extension can be a lateral extension that is not substantially moveable relative to the base in a manner that certain types of known self-fixating tip extensions are moveable, for instance between a non-deployed or non-extended position that places an extension against the base to allow insertion of the self-fixating tip into tissue with a reduced size or shape profile, and a deployed or extended position that places the extension away from the base to engage tissue and prevent movement of the self-fixating tip in a direction opposite of the direction of insertion. Alternate embodiments of lateral extensions can be moveable or deflectable, if desired, such as to allow a reduced insertion force by use of lateral extensions that deflect backward when a self-fixating tip is being pushed through tissue.

In the specific example of a self-fixating tip 16 for insertion to tissue of the levator ani, an exemplary length of a lateral extension can be a length that is less than the total thickness of levator ani tissue; a length of a lateral extension intended to be inserted into the e.g., puborectalis muscle can be a length that is a portion of the thickness of the puborectalis muscle, e.g., less than 1 centimeter, such as less than 0.5 centimeter.

As noted, a self-fixating tip 16 can include multiple lateral extensions at multiple locations, either at different positions along a length of a base, at different locations around a perimeter of a base, or both. With self tissue anchor of reduced dimensions (to achieve functionality as described), a self tissue anchor may preferably include all lateral extensions originating from the same position along a length of a base, e.g., a single set of lateral extensions can be arranged around a perimeter of a base, each extending in a different direction but from the same portion of length between the proximal base end and the distal base end.

According to preferred methods of the invention, a self-fixating tip 16 may be placed into pelvic tissue that is a fibrous tissue such as muscle, with specific examples including the levator ani muscles. Preferably, an elongate portion of an insertion tool 40 can include an engagement surface for contacting a self-fixating tip 16, the engagement surface being in the form of any one of an internal channel or an external surface, channel, extension, or other structure.

According to an aspect of the invention, an implant 10 can include one or more self-fixating tips 16 at ends of extension portions 14, and an implantation method can include placing the self-fixating tips 16 within tissue in the pelvic region to support the implant 10 as the implant 10 supports a type of pelvic tissue. The tissue can be a fibrous tissue such as a muscle of the levator ani (e.g., puborectalis, pubococcygeous, and iliococcygeous).

Still referring to FIG. 7, implant 10 may include a first self-fixating tip 16a, a second self-fixating tip 16b, a first extension portion 14a, a second extension portion 14b, and a tissue support portion 12. As illustrated, tissue support portion 12 may be suspended between first extension portion 14a and a second extension portion 14b and may be operably attached to a first end of each self-fixating tip 16a, 16b. The overall dimensions of the implant 10 may be 6-15 cm in length, in the range from 6 to 10, 8 to 10, 10 to 15, 10 to 12, or 12 to 15 centimeters in length, and 1-2 cm, more preferably 1-1.5 cm, in width (at the arms). The total length dimension between anchors should be at least sufficient to extend from a levator ani muscle on one side of the anus and into a levator ani muscle on the opposite side of the anus. Self-fixating tips 16a, 16b include a base, optional internal channel (not shown), and two lateral extensions, and the design is to allow self-fixating tips 16 to be implanted securely within tissue of the levator ani.

FIG. 7 includes a perspective view of one implant embodiment of the present invention, and the invention is not limited to the particular embodiment shown. It is understood that a large number of different sizes, shapes, and dimensions of implant (e.g., implants) will be suitable according to different embodiments of methods and implants described herein. In one embodiment the implant 10 and extension portion 14a, 14b are all substantially one piece (i.e., “integrated”) and may be of uniform width and thickness. In such an embodiment the implant may appear as one continuous ribbon or tape. In further embodiments, implant 10 may be an assembly of two or more pieces, e.g., different pieces of mesh or combinations of mesh and a biologic material.

Implant 10 may be made by being woven, knitted, sprayed, or punched from a blank. In one aspect of the invention, implant 10 may include one or more woven, knitted, or inter-linked filaments or fibers that form multiple fiber junctions. The fiber junctions may be formed via weaving, knitting, braiding, or through other techniques, including combinations thereof. In addition, the size of the resultant openings or pores of the mesh may be sufficient to allow tissue in-growth and fixation within surrounding tissue.

The material used to make the tissue support portion 12, extension portions 14a, 14b, and self-fixating tips 16a, 16b, may include a variety of different plastics or other materials that are strong but conducive to being used in the body, such as, but not limited to, polypropylene, cellulose, polyvinyl, silicone, polytetrafluoroethylene, polygalactin, Silastic, carbon-fiber, polyethylene, nylon, polyester (e.g. dacron) PLLA, acetols, EPTFE and PGA. Tissue support portion 12, extension portions 14a, 14b, and self-fixating tips 16a, 16b, each may independently be any of resorbable, absorbable or non-absorbable; optionally, some portions may be absorbable and other portions may be non-absorbable. In further embodiments the material used to make the tissue support portion 12 may include a non-synthetic material or a synthetic and non-synthetic blend of materials. In addition, it may be preferable that the tissue support portion 12 be relatively elastic. In other embodiments the implant may be relatively inelastic.

Some example of commercially available materials may include MarleX™ (polypropylene) available from Bard of Covington, R.I., Prolene™ (polypropylene) and Mersilene (polyethylene terephthalate) Hernia Mesh available from Ethicon, of New Jersey, Gore-TeX™ (expanded polytetrafluoroethylene) available from W. L. Gore and associates, Phoenix, Ariz., and the polypropylene implant available in the SPARC™ implant system, available from American Medical Systems, Inc. of Minnetonka, Minn. Commercial examples of absorbable materials include Dexon™ (polyglycolic acid) available from Davis and Geck of Danbury, Conn., and Vicryl™ available from Ethicon.

First and second extension portions 14a, 14b may likewise be made by weaving, knitting or in any of the other ways previously discussed in reference to tissue support portion 12. Extension portions 14a, 14b may be made of the same or different material as tissue support portion 12 and may include the same or different physical characteristics, such as, for example, reabsorbability. In one embodiment, first and second extension portions 14a, 14b may be a weave that results in a stronger or denser material than the weave used to make the tissue support portion 12 so as to support more weight over a given surface area. In further embodiments, tissue support portion 12 and the first and second extension portions 14a, 14b may be made of one continuous weave structure of the same or different weave densities.

FIG. 8 illustrates an alternate embodiment of an implant of the invention. Self-fixating tips 16a, 16b are at ends of extension portions 14a, 14b, which are each connected at their opposite ends to a soft tissue support portion 12. Self-fixating tips 16a, 16b may be placed at a muscle of the levator ani, such as the puborectalis muscle, pubococcygeous muscle, or illiococcygeous muscle. As illustrated, implant 10 can be a biologic material, but could alternately be a mesh or other synthetic material. Extension portions 14 are illustrated to be of synthetic mesh.

Optionally, according to various implant embodiments, a material that forms any portion of an implant 10 may include one or more substances incorporated into the material or coated onto the material of the implant. Examples of substances may include, without limitation, drugs, hormones, antibiotics, antimicrobial substances, dyes, silicone elastomers, polyurethanes, radiopaque filaments or substances, position or length indicators, anti-bacterial substances, chemicals or agents, including any combinations thereof. A substance or material may be used to enhance treatment effects, reduce potential implant rejection by the body, reduce the chances of tissue erosion, allow or enhance visualization or location monitoring, indicate proper implant orientation, resist infection, or other provide other desired, useful, or advantageous effects.

Once a first anchor 16a is placed into a desired position, a second anchor 16b may be inserted through the same incision and placed in a desired position on an opposite side of the patient. As with the first self-fixating tip 16a, the second self-fixating tip 16b may be positioned with or without the assistance of an introducer and may be placed, e.g., into tissue of the levator ani (puborectalis muscle, pubococcygeous muscle, or illiococcygeous muscle). Tissue support portion 12 may be properly oriented into the desired position in relation to the anus. It may be desirable to ensure that the implant 10 is not twisted during implantation. Positioning of the implant 10 can be accomplished by selecting the point of entry and depth of each anchor 16.

As an example, implants for treating incontinence can include a portion useful to support the rectum to address anal or fecal incontinence. For example an implant 10 is used exclusively to support the anal sphincter, and may be in the form of a mesh strip that includes a support portion 12 implanted below or at the rectum. A preferred distance between distal ends of extension portions 14a, 14b designed to support the rectum can be of a total length between distal ends (e.g., self-fixating tips 16) to allow the combined length of extension portions 14 and tissue support portion 12 to extend from a right levator ani to a left levator ani, e.g., from one puborectalis muscle to the other puborectalis muscle. This length is shown at FIG. 7 as length L1 between self-fixating tips 16a, 16b (the length excluding the length of both of the self-fixating tips), and as length L2 including both self-fixating tips 16a, 16b. Useful lengths of extension portions are as desired, and are exemplified elsewhere in the present description.

According to embodiments of implants and methods, a fixed-length implant or implant portion (e.g., as exemplified in FIGS. 7, 8, and 10), including no length-adjusting mechanism, can be placed with desired positioning and effect (e.g., supportive force, approximation, or both) on pelvic tissue, by selective placement of self-fixating tips 16a, 16b within pelvic tissue. The implant 10 and self-fixating tip 16 can exhibit desirable “adjustability” or “positionability” features, without the need for a length-adjusting mechanism, as follows. Each self-fixating tip 16 of a implant 10 can be placed within a pelvic tissue such as tissue of the levator ani, with properties of self-fixating tips 16 (e.g., dimensions, pullback force, number of lateral extensions) and implant 10 (dimensions such as length between self-fixating tip) being sufficient to allow this placement at tissue on one or both sides of the pelvic region (e.g., at opposing levator ani), while the tissue support portion 12 of the implant 10 supports the rectum, anus, etc. Desired position of the implant 10, the amount of approximation of the supported tissue (e.g., anal sphincter), or the amount of supportive force placed on the supported tissue, can be achieved by selecting the placement of the self-fixating tips 16. Placement can include the position at which the self-fixating tip 16 is inserted into tissue (the point of insertion) such as the placement of a self-fixating tip 16 within tissue of the levator ani and (if the pelvic tissue includes sufficient depth) can also include the depth to which the self-fixating tip 16 is placed into pelvic tissue such as a muscle of the levator ani. Each of the point of insertion, and depth of insertion, can be separately selected to result in a desired position of the implant, tension on the implant, approximation of pelvic tissue, or supportive force applied to pelvic tissue to be supported by the implant.

FIG. 8 demonstrates an alternative embodiment of implant 10. Implant 10 has a narrower width of extension portions 14 and a wider width of tissue support portion 12. A wider tissue support portion 12 sits under the rectum and is capable of distributing forces over a wide area.

FIG. 9 illustrates an implant 10 comprised of multiple pieces, for example three pieces. The tissue support portion 12 is held in place under the rectum by one or more extension portions 14 placed through the levator ani with a self-fixating tip 16. The one or more extension portions 14 have a self-fixating tip 16 operatively coupled to the distal end of the extension portion 14. The one or more extension portions 14a, 14b may operatively connect to the tissue support portion 12, providing the surgeon the possibility of adjusting the tension and length of the extension portions 14a, 14b.

In another embodiment of an implant according to the invention, an implant for treating anal or fecal incontinence, may be assembled from separate pieces, e.g., as a modular assembly of parts, which can be advantageous for reasons including flexibility in placement of the different pieces and in sizing of an assembled modular implant. FIG. 9 illustrates implant 10 that includes a single tissue support portion 12, and two extension portions 14a, 14b each including a self-fixating tip 16a, 16b (as described herein). Once each extension portion 14 is placed, extension portions 14 may be attached at their connecting ends (end opposite self-fixating tip) by use of any fastening mechanism, such as by clips, sutures, or other methods known to those in the art or developed in the future to tissue support portion 12.

In still yet another embodiment of a multiple piece implant, implant 10 comprises of two pieces. As illustrated in FIG. 4b, a first piece includes a tissue support portion 12, extension portion 14, and self-fixating tip 16, and a second piece includes a tissue support portion 12, extension portion 14, and self-fixating tip 16. The tissue support portions 12 can attach to the anal sphincter or surrounding tissue on a first and second side. Each self-fixating tip is connected to an end of the extension portion 14 and extends to the levator ani to provide support to the tissue.

FIGS. 11a and 11b illustrate a device to aid in keeping the posterior incisions open. Tape 60 may be used as a means for separating the posterior incisions 62 to provide access to the posterior incisions 62 during a implant insertion procedure. In one embodiment, tape 60 may aid in keeping a single posterior incision open as shown in 11a. Alternatively, tape 60 may aid in keeping two posterior incisions open as shown in FIG. 11b. The tape 60 may have an adhesive that sticks to tissue on at least one side.

An embodiment of a kit according to the invention includes an insertion tool 40 and an implant 10. Implant 10 can be installed to help maintain fecal continence by supporting the rectum to restore the ano-rectal angle. The present invention also includes methods of implanting the implant 10 by implant through a posterior incision, and attached to (e.g., anchored to) the levator ani muscle on either side of the anus. Only requiring a posterior incision to the anus eliminates additional incisions such as external incisions used in some methods of implanting other implants, along with the scarring and invasiveness associated with the extra incisions. It also eliminates the need for anterior incisions, such as those located at tissues such as vaginal, abdominal, perineal, perirectal, or transobturator. Implant 10 and its methods of implantation are, therefore, a reduced or “minimally” invasive treatment option for patients suffering from anal or fecal incontinence. The present disclosure may focus on the puborectalis muscle or pubococcygeous muscle anchoring location, with the understanding that other anchoring locations may be selected by those of skill in the art.

An insertion tool 40 can be used to install the implant. Various types of insertion tools are known, and these types of tools and modifications thereof can be used according to this description to install an implant. Examples of useful tools include those types of tools that generally includes a curved elongate needle 44 and a handle 42; a handle 42 attached to one end (a proximal end) of the needle 44; and a distal end of the needle 46 is adapted to engage a self-fixating tip 16 that allows the needle 44 to push the self-fixating 16 through a tissue passage and insert the self-fixating tip 16 within tissue of the pelvic region. This class of tool can be used with a self-fixating tip 16 that includes an internal channel designed to be engaged by a distal end of an insertion tool 46. Other general types of insertion tools 40 will also be useful, but may engage a self-fixating tip 16 in a manner that does not involve an internal channel of a self-fixating tip 16. These alternate insertion tools may for example contact or grasp a proximal base end of a self-fixating tip 16 in the absence of an internal channel extending from the proximal base end toward the distal base end, such as by grasping an external surface of the base.

Exemplary insertion tools for treatment of incontinence are described, e.g., in U.S. Pat. Nos. 7,070,556 and 7,740,576; and PCT application numbers 2006/028828; 2006/0260618; and 2013/0006048. Tools described in those patent documents are designed for placement of an implant in a pelvic region for the treatment of prolapse, male or female incontinence, etc. Other described insertion tools include a two-dimensional elongate needle that allows a user to place an extension portion of an implant through a posterior incision to a region of levator ani tissue.

Exemplary insertion tools 40 for use according to the invention can be similar to or can include features of tools described in the above-referenced patent documents. For use according to methods described herein, those insertion tools 40 may be modified to allow the insertion tool 40 to be used to place a self-fixating tip 16 at tissue within the pelvic region through a tissue path that does not extend to an external incision. The insertion tool 40 can be designed, shaped, and sized, to include an elongate inserter or needle 44 that may be straight or that may be curved in two or three dimensions, that can be inserted through a posterior incision, and to extend from that incision to a pelvic tissue location for placement of a self-fixating tip.

According to certain embodiments, an internal channel of a self-fixating tip 16 can fit over a length of a distal end of a needle of an insertion tool 40 with a single or alternate fixed radial orientation relative to an axis of an insertion tool 40.

Thus, an example of a combination of insertion tool 40 and self-fixating tip 16 according to this description can include an elongate curved needle 44, hollow tube, or other “elongate inserter,” curved in two dimensions, and a self-fixating tip 16; the distal end of the needle 46, tube, or inserter, and the self-fixating tip 16, include complementary engaging surfaces that can cause the self-fixating tip 16 to be oriented at the distal end of the needle 44, tube, or inserter so that lateral extensions are oriented to be perpendicular (90 degrees, or more broadly, at an angle in the range from 80 to 100 degrees) to a plane defined by the two-dimensional curve.

The needle 44 may be of a length that allows the end of the insertion tool 40 to be inserted through a posterior incision and to reach a puborectalis, pubococcygeous or illiococcygeous muscle. The insertion tool 40 can be useful for placing a self-fixating tip 16 at tissue of the levator ani, or other tissue of the pelvic region, preferably with lateral extensions being oriented non-parallel to fibers of a fibrous tissue.

Insertion tool 40 may be any type of tool that can engage self-fixating tip 16 to drive it through and into pelvic tissue of a desired location. Such an insertion tool 40 may include a durable biocompatible, curved or straight needle portion 44, made, e.g., of stainless steel, titanium, Nitinol, polymers, plastics, or other individual or combinations of materials. Handle 42 is attached at a proximal end of needle portion 46, and distal end of needle portion 16 is designed to engage self-fixating tips 16, e.g., by being sized and shaped to fit within an interior channel of each tip. Insertion tool 40 should have sufficient structural integrity to position self-fixating tip 16 as desired. Insertion tool 16 may mate with or engage self-fixating tip 16 by any manner, including fitting within an internal channel of a body or base of tip 16, alternately on an external portion of a body or base of a self-fixating tip 16, or by interacting with the lateral extensions. Self-fixating tip 16 may be situated inside or outside of insertion tool 40.

Any releasable engagement and detent mechanism that is capable of holding a self-fixating tip 16 at a distal end of an insertion tool 40 may be useful according to the present description. As will be appreciated, a number of different structures, mechanisms, collars, locking arms, or other mechanical features may be integrated into an insertion tool 40. A detent or other releasable attachment between a distal end of an insertion tool 40 and a self-fixating tip 16 may operate on principles of a friction fit, a snap fit, a twist connection, a rotating connection, a moveable engagement, or any other structure of method known to those in the mechanical engagement, holding, and release arts. The engagement may contact any portion of a self-fixating tip 16, such as an internal bore of a base, an external surface of a base, a lateral extension, etc.

In an alternative embodiment, insertion tool 40 comprises a handle 42 that further comprises a release button or trigger 48. Upon activation of the release button 48, the self-fixating tip 16 is deployed from the insertion tool 40. In addition, the handle 42 further includes a safety lock (not shown) to prevent premature ejection or release of the self-fixating tip 16. The release button or trigger 48 within the handle 42 can include a cam or follower mechanism causing a sheath 50 to rotate around the needle shaft, such that the anchor 16 is caused to move longitudinally in a distal direction off of the end of the distal end portion 52.

The needle 44 can further include a rotatable sheath or tube 50 and a distal end portion 52. In various embodiments, the needle 44 can be hollow, solid, curved, straight, helical, or can take on a myriad of other like and compatible configurations. The sheath 50 generally shrouds a length of the needle 44. The distal end portion 52 can include an anchor retention and deployment tip 54. The release button or trigger 48 is configured to be in operable communication with the sheath 50.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method for treating anal incontinence, the method comprising:
creating a posterior incision posterior to an anus,
providing an implant comprising a tissue support portion, extension portion, and a self-fixating tip,
providing an insertion tool,
engaging the insertion tool with the self-fixating tip,
inserting the insertion tool and self-fixating tip through the incision, and
inserting the self-fixating tip into tissue of a levator muscle.

2. The method of claim 1, wherein the implant further comprises at least one extension portion.

3. The method of claim 1, wherein the posterior incision is a single incision medial to the anus.

4. The method of claim 1, wherein the posterior incision is two incisions lateral to the anus.

5. The method of claim 1, wherein the tissue of the levator muscle is puborectalis, pubococcygeous, or illiococcygeous.

6. The method of claim 1, wherein the implant comprises two tissue support portions, two extension portions, and two self-fixating tips.

7. The method of claim 1 wherein the tissue support portion is wider than the extension portions.

8. The method of claim 2, wherein the pelvic implant comprises two extension portions.

9. The method of claim 8, wherein the pelvic implant comprises a self-fixating tip at each extension portion end.

10. The method of claim 1, wherein the extension portion is adjustably connected to the tissue support portion.

11. The method of claim 1 comprising:
creating a single incision posterior to an anus;
dissecting tissue beneath an anal sphincter;
implanting the self-fixating tip at the levator muscle on one side of a patient;
implanting the self-fixating tip at the levator muscle on an opposite side of the patient, whereby the implant is suspended between the levator muscles; and
positioning a portion of the implant around the anal sphincter.

12. The method of claim 1 comprising:
creating a first posterior incision lateral to the anus on one side of a patient;
creating a second posterior incision lateral to the anus on an opposite side of a patient;
dissecting tissue beneath an anal sphincter;
implanting the self-fixating tip at the levator muscle on one side of a patient;
implanting the self-fixating tip at the levator muscle on the opposite side of the patient, whereby the implant is suspended between the levator muscles; and
positioning a portion of the implant around the anal sphincter.

13. The method of claim 1, wherein the method does not require an anterior incision selected from a perineal incision, vaginal incision, abdominal incision, and an obturator foramen incision.

14. A kit comprising an implant and an insertion tool, the implant comprising a tissue support portion, an extension portion, and a self-fixating tip, wherein the implant is adapted to extend from a region of anal musculature to a region of a levator ani to support the anal musculature.

15. The kit of claim 14, wherein the implant is adapted to extend from a region of levator ani on a patient's first side to a region of levator on a patient's opposite side and under the anal musculature to support the anal musculature.

16. The kit of claim 14, wherein the insertion tool comprises an actuator mechanism and an elongate sheath, wherein the actuator mechanism is in operable communication with the elongate sheath such that activation of the actuator operably rotates the sheath relative to the needle to deploy the self-fixating tip.

17. The kit of claim 14, wherein the extension portion is adjustably connected to the tissue support portion.

18. The kit of claim 14, wherein the implant comprises two pieces, each piece comprising a distal end and a proximal end, the self-fixating tip located at a distal end, the tissue support portion at a proximal end, and an extension portion located in between.

19. The kit of claim 14, wherein the implant comprises two extension portions, and two self-fixating tips, each extension portion having a distal end and a proximal end, and the tissue support portion located between the proximal ends of each extension portion, and each self-fixating tip at the distal end of each extension portion.

20. The kit of claim 14, wherein the insertion tool comprises a handle and a needle, wherein the needle includes a distal tip adapted for selective engagement with the self-fixating tip.
